(19) United States
(12) Patent Application Publication
(10) Pub. No.: US 2022/0054446 A1
(43) Pub. Date: Feb. 24, 2022

(54) TARGETED TREATMENT WITH A TOPICAL CREAM OF CANNABINOIDS, TERPENES, AND ESSENTIAL OILS

(71) Applicants: Yvette Rose Webb, Hamilton (CA); Clive Alan Webb, Hamilton (CA)

(72) Inventors: Yvette Rose Webb, Hamilton (CA); Clive Alan Webb, Hamilton (CA)

(21) Appl. No.: 16/996,835

(22) Filed: Aug. 18, 2020

(51) Int. Cl. A61K 31/352; A61K 33/06; A61K 31/05; A61K 31/015; A61K 31/045; A61K 36/67; A61K 36/537; A61K 36/53; A61K 9/06

(52) U.S. Cl. CPC A61K 31/352; A61K 33/06; A61K 31/05; A61K 31/015; A61K 9/06; A61K 36/67; A61K 36/537; A61K 36/53; A61K 31/045

(57) ABSTRACT

Prior art describes methods for making whole-plant extracts from the Cannabis plant. Terpenes and cannabinoids are classes of compounds found inherently in those extracts. But, because those extracts contain hundreds of different, and sometimes counteracting, compounds, they are not the most effective treatments for common health issues. Specific terpenes and cannabinoids may be isolated and chosen for their use for specific conditions. Examples of the present invention differ from the prior art by selecting isolated compounds (Like the cannabinoid THC and the terpene Myrcene) and specifically combining them with essential and topical agents to produce targeted treatments.

### FIELD OF THE INVENTION

The present invention is directed to a topical cream comprising cannabinoids, terpenes, and essential oils, which can be used to treat illness, disease, or other health conditions. Cannabinoids, like terpenes, are compounds found in the Cannabis plant and may be used in the treatment of different illnesses. While terpenes and cannabinoids, are specific compounds extracted from Cannabis, essential oils are extracts from other plants (like lavender or black pepper) which contain many different compounds. When chosen correctly and used together, terpenes, cannabinoids, and essential oils can have a significant positive impact on many common health conditions, such as, but not limited to, pain, inflammation, anxiety, and depression.

### SUMMARY OF THE INVENTION

Cannabis has been used for medicinal purposes for millennia. The Cannabis plant contains many compounds, each of which have differing effects. The most commonly known compound in Cannabis is THC, a psychoactive substance that effects the body's endocannabinoid system. Cannabis contains additional cannabinoids, like CBD, which has grown in renown over the last few decades as its medicinal applications have been further investigated. Essential oils are compounds extracted by processing plants to create an odor that replicates the ‘essence’ of plants and are usually named after the plant from which they were derived. Essential oils are routinely used in aromatherapy to treat various conditions. Essential oils contain various hydrocarbons and terpenes. Terpenes are a class of organic compounds, which usually have a strong odor, and are produced by a variety of plants and some insects. Many different terpenes may be found in a single plant, as is the case for the many terpenes that can be found in the Cannabis plant. Extensive research has been done to determine what effects each terpene produced by the Cannabis plant may have on the body. Yet, there remains a need for targeted topical treatments made from compositions of terpene isolates, essential oils, and specific cannabinoids.

### SUMMARY

Embodiments of the present invention include topical creams, with one embodiment being comprised of a base cream, THC, CBD, Caryophyllene, Linalool, Myrcene, lavender essential oil, rosemary essential oil, black pepper essential oil, and magnesium chloride lotion; this embodiment may be used to treat nighttime leg cramp pain. Another embodiment of the present invention is comprised of THC, CBD, Caryophyllene, Linalool, Myrcene, clary sage essential oil, rosemary essential oil, and black pepper essential oil; this embodiment may be used to treat chronic pain. Additional embodiments of the present invention further comprise additional therapeutic compounds to treat a range of conditions.

### DETAILED DESCRIPTION

Cannabis is a genus of flowering plants in the family Cannabaceae. As used herein, Cannabis includes Cannabis sativa, Cannabis indica, and Cannabis ruderalis. Over the last century, there has been a shift in views about the usefulness of Cannabis. Many developed nations outlawed the use of the plant, recreationally and medically. In recent decades, restrictions on the use of Cannabis have been eased, and intense scientific study of the plant's medicinal properties has been allowed. Whereas recreational use of Cannabis often refers to smoking dried components of the plant (like the flower of the plant) to achieve a high, medical use of Cannabis (medical Cannabis or medical marijuana) refers to the use of Cannabis and its constituent parts to treat diseases or improve symptoms. Medical Cannabis, in a majority of instances, involves utilizing the dry flower of the plant, either for smoking and inhalation or to produce infusions for oils and edibles. Recent developments in the field of medical Cannabis involve extracting and isolating specific compounds from the plant and using those compounds for treatments, rather than utilizing the dry flour of the plant. The two most commonly referenced groups of compounds that may be extracted and isolated from Cannabis are cannabinoids and terpenes.

Cannabinoids are a group of compounds found in glandular trichomes on the Cannabis plants; when consumed, these compounds produce mental and physical effects on the human body. The body reacts to phytocannabinoids, which are cannabinoids derived from plants (like Cannabis), similarly to how it reacts to endocannabinoids, which are endogenous cannabinoids produced naturally by the body. The two most studied cannabinoids derived from Cannabis are THC (tetrahydrocannabinol) and CBD (cannabidiol). Both of these compounds interact with the body's endocannabinoid system and may affect the release of neurotransmitters. The interaction of THC and CBD with the body is complex, and the two cannabinoids may produce a synergistic effect. For example, THC binds with the cannabinoid 1 (CB1) receptors in the brain and produces a high or sense of euphoria. CBD binds weakly to CB1 receptors and may interfere with the binding of THC and dampen its psychoactive effects. Known uses for CBD include, but are not limited to, pain relief, nausea reduction, migraine frequency reduction, migraine pain reduction, anxiety reduction, depression easing, seizure frequency reduction, anti-inflammatory treatment, insomnia treatment, and psychosis treatment. Known uses for THC include, but are not limited to, pain relief, nausea reduction, migraine frequency reduction, migraine pain reduction, anxiety reduction, anti-inflammatory treatment, insomnia treatment, and appetite promotion.

Terpenes are another group of compounds found in the glandular trichomes of the Cannabis plant which generate mental and physical effects when consumed. As used herein, the term, terpenes, also includes terpenoids. Terpenes are fragrant oils that give Cannabis its aromatic diversity. They are what give certain strains of Cannabis their smell: for example, terpenes give the blueberry strain its signature berry smell, and the lavender strain its sweet floral aroma. These fragrant oils are secreted in Cannabis's sticky resin glands, the same ones that produce THC, CBD, and other cannabinoids. Terpenes are not unique to Cannabis; they can be found in many other herbs, fruits, plants, and insects. Cannabis plants contain over 100 different terpenes, including but not limited to, Myrcene, Limonene, Linalool, Caryophyllene, Pinene, Levomenol (Bisabolol), Eucalyptol, Nerolidol, Carene (Delta-3-Carene), Camphene, Borneol, Terpineol, and Geraniol.

Myrcene is a common terpene and is found in many different Cannabis strains. The aroma of myrcene is similar to cloves. Myrcene may be used as an analgesic, pain reliever, anti-inflammatory, and antibiotic. Myrcene is believed to block the action of cytochrome, aflatoxin B, as well as carcinogens. Myrcene also has a relaxing or sedative effect, and when combined with THC, may increase the psychoactive potential and absorption rate of the cannabinoid through a synergistic effect. The synergistic effect of Cannabis-derived-terpenes with cannabinoids is commonly known as the entourage effect. Multiple studies have demonstrated how the entourage effect works in combinations of Cannabis-derived-terpenes and cannabinoids.

Limonene is also a terpene found in abundance in many Cannabis stains. The aroma produced by limonene is a citrus-like smell, similar to that of a lemon. Limonene is largely used in alimentary items, cleaning products, and it is one of the most frequent fragrances used in cosmetics formulation. Limonene is believed to improve one's mood, reduce depression, reduce stress and anxiety. Limonene may also possess antifungal, anticarcinogenic, and antibacterial properties. Limonene may also prevent the deterioration of the RAS gene, which is one of the factors that contributes to the development of tumors. This terpene may also protect against Aspergillus and other carcinogens present in smoke, as well as boost the immune system. Limonene is able to easily penetrate the blood-brain barrier and increase systolic pressure, resulting in increased attention, focus, mood, and libido. Studies have shown additional therapeutic effects of limonene, such as, anti-inflammatory, antioxidant, antinociceptive, anticancer, antidiabetic, antiviral, and gastroprotective effects. Scientific literature regarding the usage of limonene and its activities and underlying mechanisms involved in combating diseases is still being investigated and reviewed to determine efficacious targeted treatments.

Caryophyllene is a unique terpene, and it the only terpene known to bind directly to the cannabinoid receptors. Caryophyllene produces an aroma similar to pepper or spice. This terpene binds selectively to CB2 receptors. Binding to CB2 receptors contributes to caryophyllene's effectiveness in uses like as an anti-inflammatory, anti-fungal, analgesic, and to reduce anxiety. More recent studies have suggested that caryophyllene may be useful in treating substance abuse disorders, like alcohol use disorder.

Pinene is the terpene responsible for the familiar smell associated with pine and fir trees. It is the main ingredient of the essence of turpentine. It is also present in many plants such as sage. Pinene is used in medicine as an expectorant, bronchodilator, anti-inflammatory and local antiseptic. It also crosses the blood-brain barrier very easily, where it acts as an inhibitor of acetylcholinesterase, preventing the destruction of molecules responsible for the transmission of information, which results in memory improvement. This terpene also counteracts some of the effects of THC, which leads to a decrease in the acetylcholine levels. While THC may inhibit memory, a mixture of THC and pinene may not inhibit memory. Pinene also improves concentration, personal satisfaction, and energy. Pinene has an anti-inflammatory effect on humans. But more importantly, pinene helps improve airflow and respiratory functions, while also helping to reduce memory loss related to THC. Studies have shown that this terpene can actually help with asthma. Additional uses of pinene include use for the treatment of arthritis, Crohn's disease, and cancer.

Terpineol is a terpene with an aroma similar to lilac, crabapple blossoms or lime blossoms. This terpene is commonly added to perfume and other cosmetics for its aroma, though studies suggest that terpinol has many beneficial effects on the body, including antibiotic and antioxidant effects. Additionally, studies on mice have shown that terpineol also has a sedative effect that reduces mobility.

Borneol is a terpene with an aroma similar to mint. This terpene acts as a natural insect repellant but may also be used to treat human illnesses. Traditional Chinese medicine uses borneol to treat fatigue and stress. Modern studies have tested the anti-cancer properties of this terpene, specifically its efficacy in killing mutated cells in breast cancer patients.

Linalool is a terpene found in abundance in many plants and has an aroma with spicy and floral notes. Linalool may be used to treat various conditions, like epilepsy or insomnia by producing a sedating and relaxing effect on the body. Studies suggest that this terpene also reduces anxiety and elevates mood.

Camphene is terpene with a pungent aroma similar to fir needles or damp soil. Although this terpene is considered a minor terpene in the Cannabis plant, the potential health benefits of this isolated compound are major. Studies suggest that camphene is effective in treating cardiovascular disease, and as due to its antioxidant properties, is also able to reduce oxidative stress on cells. Camphene may also reduce inflammation.

Unlike terpenes and cannabinoids (which are isolated compounds which may be derived from plants), essential oils are whole-plant extracts, meaning they are a mixture of many different compounds found naturally in either the whole plant or one component of a plant. Essential oils are named after the plant from which they are derived, and include, but are not limited to, clary sage essential oil, lavender essential oil, rosemary essential oil, black pepper essential oil, pepper mint essential oil, lemon essential oil, and orange essential oil. The process of making essential oils is well known in the art and usually involves distillation or cold pressing of the plant, followed by the addition of a carrier oil to the extracted compounds. The compounds contained within each essential oil are numerous and include, but are not limited to, terpenes, acids, and alcohols. While studies have been conducted to determine the effects of the isolated compounds within essential oils, extensive research on the effects of the essential oils themselves has also been completed, as the traditional use of essential oils in aromatherapy is to utilize all of the compounds in the oils. An individual essential oil, for example lavender essential oil, may differ in its chemical composition based on where and how the plant was grown, as well as the variety of the plant within the species. Essential oils contribute significantly to affecting the mood of the user, which is a prominent feature of essential oils. However, many studies have also shown essential oils to be effective in treat various health conditions.

Lavender essential oil is derived from lavender plants and has many known health benefits. Lavender essential oil has been shown to elevate mood and decrease anxiety, and these effects have been shown in use of the oil in aromatherapy as well as its use as an additive in dietary supplements. Lavender essential oil also treats insomnia by promoting sleep, and these effects have been demonstrated in a variety of populations: from college students to nursing home patients. This oil is also purported to have anti-inflammatory, antifungal, antiseptic, antibacterial, and antimicrobial properties, as well as antispasmodic, analgesic, detoxifying, hypotensive, and sedative effects.

Rosemary essential oil is derived from rosemary plants and has many known health benefits. Rosemary essential oil has been shown to reduce stress and ease anxiety and increase mood by easing mental strain and preventing fatigue; these effects are especially evident when measuring stress, anxiety, and mood in the short-term. Additional studies have demonstrated that rosemary essential oil may increase brain function by improving memory, as well as relieve pain. Rosemary essential oil has also been shown to promote hair growth, increase blood circulation, and reduce inflammation.

Clary sage essential oil is derived from clary sage (Salvia sclarea) plants and has many known health benefits. This oil acts as a natural antidepressant by increasing cortisol levels. There is also evidence to suggest that clary sage essential oil has some antibacterial properties. Recent studies have also demonstrated that this essential oil reduces pain, with its most promising effect being its ability to reduce menstrual pain from cramps.

Black pepper essential oil is derived from black pepper (Piper nigrum) plants and has many known health benefits. Black pepper essential oil elevates mood and decreases anxiety, but this oil's mood-altering effects do not end there, as studies have also shown that it reduces addiction cravings, most notably cigarette (nicotine) cravings. This essential oil also stimulates appetite. Due to its anti-inflammatory and antispasmodic properties, black pepper essential oil works to reduce muscle injuries, tendonitis, and symptoms of arthritis and rheumatism. Although the precise impacts of black pepper essential oil on pain requires further research, studies have already shown that this oil is able to reduce pain.

Prior art describes methods of producing concentrates and extracts from the Cannabis plant. Concentrates are produced using mechanical processes to accumulate the Cannabis trichomes and their contents. Extracts are produced using solvents and distillation to separate compounds. Extracts containing some of the plant's terpenes, and other cannabinoids are known as Broad Spectrum extracts, while Full Spectrum extracts (often called whole plant extracts) contain all the individual cannabinoids, terpenes, and other bioactive compounds found within the trichomes. Cannabis concentrates and extracts usually contain both cannabinoids and terpenes, as both cannabinoids and terpenes are found inherently in the Cannabis plant. Prior art also describes the selection or creation of certain Cannabis strains which produce specific amounts (or percentages) of cannabinoids and terpenes as well as methods of producing concentrates and extracts from these plants. Prior art also describes a type of synergistic effect, called the entourage effect. Terpenes and Cannabinoids produce an entourage effect, when both compounds are present in a composition, where the terpenes enhance certain effects that cannabinoids have on the body, thereby creating an entirely new effect that may not be achieved with either of the compounds separately. Certain embodiments of the present invention do not relate to the production of concentrates and extracts from the Cannabis plant, but rather trailblaze the art by utilizing extracted and isolated compounds that may be found in the Cannabis plant as well as utilizing whole plant extracts from completely different plants in a topical cream to treat specific health conditions.

Embodiments of the present invention include a topical cream comprising isolated cannabinoids, terpene isolates, and essential oils. Using isolated compounds in the topical cream composition allows for more flexibility and specificity in the targeting of certain health conditions, as certain cannabinoids and/or terpenes may be chosen for their desired effects. The inclusion of essential oils in the topical cream produces an additional entourage effect as the various compounds of the essential oils work synergistically with the isolated cannabinoids and terpene isolates. As used herein, the terms “cream”, “topical”, and “topical cream” are not restrictive and include in their meanings, ointments, gels, lotions, creams, salves, liniments, pastes, and unguents. As used herein, isolated cannabinoids may include, but are not limited to, isolated cannabinoids in powder, nanoparticles, or molecule form, and cannabinoid oils (like THC oil or CBD oil). As used herein, the term “isolated” in “isolated cannabinoid” refers to nature of the cannabinoid compound, being significantly isolated from other bioactive compounds, and should not be interpreted as restricting the meaning to pure isolates (examples of pure isolates are 99% CBD and 99% THC), and should not be interpreted to restrict the method used to produce the isolation (it should be noted that a common method for producing isolated cannabinoids is extraction and distillation). Because common isolated cannabinoids, like THC oil and CBD oil, may contain both the cannabinoid compound and some carrier oil, the concentration of the THC compound and CBD compound within their respective isolated forms is an important factor in embodiments of the present invention, as different concentrations have varying effects on the body. Isolated cannabinoids may be derived from the Cannabis plant, but as research evolves, could foreseeably and obviously be derived synthetically. As used herein, terpene isolates may also include, but are not limited to, terpene oils. As used herein, the term “terpene isolate” refers to nature of the terpene compound, being significantly isolated from other bioactive compounds, and should not be interpreted as restricting the meaning to pure isolates (an example of a pure isolate is 99% Pinene), and should not be interpreted to restrict the method used to produce the isolation. Because a specific terpene isolate, like Pinene isolate, may contain both the pinene compound and some carrier oil, the concentration of terpene compound within the terpene isolate is an important factor in embodiments of the present invention. Terpene isolates may be derived from the Cannabis plant or other plants, or even be derived synthetically. As used herein, “essential oils” are whole plant extracts, but do not include whole plant extracts derived from the Cannabis plant.

One embodiment of the topical cream comprises one isolated cannabinoid, one terpene isolate, and one essential oil. Additional embodiments of the topical cream comprise more that one of the various components: isolated cannabinoids, terpenes isolates, and/or essential oils. Other embodiments of the topical cream also comprise a base cream, while another embodiment comprises a base cream and a lotion. As used herein, base cream means a cream used to facilitate the addition of other active ingredients or prevent separation of the components, and the base cream may include an emulsifier or may facilitate depth of penetration for different therapy goals.

Examples of the two different preferred embodiments of the invention are shown in Table 1 and Table 2. These examples show extensively tested formulae created by the inventor which have significant effects on the body due to the entourage effect produced by the cannabinoids, terpenes, and essential oils. Table 1 is an example of a topical cream that may be used to treat nighttime leg cramp pain. This topical cream contains a base cream, isolated THC, isolated CBD, Caryophyllene isolate, Linalool isolate, Myrcene isolate, lavender essential oil, rosemary essential oil, black pepper essential oil, and a magnesium chloride lotion. In this embodiment, the isolated THC has a concentration of 27 mg/ml and the isolated CBD has a concentration of 22 mg/ml. Table 1 also shows the percentage (as measured by comparing ml of an individual component to total ml of all components in the topical) of each component within the topical cream. The embodiment of topical cream shown in Table 1 may be prepared before use to treat an illness, disease, or other health condition with the following method. (1) Prepare the magnesium chloride lotion by mixing 45 ml of magnesium chloride flakes with 45 ml of warm filtered water and mixing by hand or mechanically until the components are fully incorporated into each other. (2) Combine the following: 12.0 ml isolated THC, 12.0 ml isolated CBD, 0.5 ml Caryophyllene isolate, 1.0 ml Linalool isolate, and 1.0 ml Myrcene isolate. (3) Add the isolated cannabinoids and terpene isolates to 210.0 ml of base cream. (4) Heat the base cream, isolated cannabinoids, and terpene isolates in a water bath, with the water bath being 65 degrees Celsius. (5) Mix the base cream with the isolated cannabinoids and terpene isolates by hand or mechanically. (6) Maintain the temperature of the mixture while also mixing in 1.9 ml lavender essential oil, 0.6 ml rosemary essential oil, and 0.6 ml black pepper essential oil by hand or mechanically. (7) Cool the resulting mixture to room temperature and mix it with 45 ml magnesium chloride lotion. As used herein, mixing “by hand” means mixing using a whisk or other simple or similar tool, while mixing “mechanically” means mixing using a machine or some other similar process.

TABLE 1

| | Component | Component Percentage of |
|---|---|---|
| Component: | Amount (ml): | Total Composition: |
| Base Cream | 210 | 73.79% |
| Isolated THC | 12 | 4.22% |
| Isolated CBD | 12 | 4.22% |
| Caryophyllene Isolate | .5 | 0.17% |
| Linalool Isolate | 1 | 0.35% |
| Myrcene Isolate | 1 | 0.35% |
| Lavender EE | 1.9 | 0.67% |
| Rosemary EE | .6 | 0.21% |
| Black Pepper EE | .6 | 0.21% |
| MgCl Lotion | 45 | 15.81% |

Table 2 is an example of a topical cream that may be used to chronic pain. This topical cream contains a base cream, isolated THC, isolated CBD, Caryophyllene isolate, Linalool isolate, Myrcene isolate, clary sage essential oil, rosemary essential oil, and black pepper essential oil. In this embodiment, the isolated THC has a concentration of 27 mg/ml and the isolated CBD has a concentration of 22 mg/ml. Table 2 also shows the percentage (as measured by comparing ml of an individual component to total ml of all components in the topical) of each component within the topical cream. The embodiment of topical cream shown in Table 2 may be prepared before use to treat an illness, disease, or other health condition with the following method. (1) Combine the following: 12.0 ml isolated THC, 12.0 ml isolated CBD, 1.0 ml Caryophyllene isolate, 1.0 ml Linalool isolate, and 0.5 ml Myrcene isolate. (2) Add the isolated cannabinoids and terpene isolates to 210.0 ml of base cream. (3) Heat the base cream, isolated cannabinoids, and terpene isolates in a water bath, with the water bath being 65 degrees Celsius. (4) Mix the base cream with the isolated cannabinoids and terpene isolates by hand or mechanically. (5) Maintain the temperature of the mixture while also mixing in 1.9 ml clary sage essential oil, 0.6 ml rosemary essential oil, and 0.6 ml black pepper essential oil by hand or mechanically. (6) Cool the resulting mixture to room temperature.

TABLE 2

| | Component | Component Percentage of |
|---|---|---|
| Component: | Amount (ml): | Total Composition: |
| Base Cream | 210 | 87.65% |
| Isolated THC | 12 | 5.01% |
| Isolated CBD | 12 | 5.01% |
| Caryophyllene Isolate | 1 | 0.41% |
| Linalool Isolate | 1 | 0.41% |
| Myrcene Isolate | .5 | 0.21% |
| Rosemary EE | .6 | 0.25% |
| Black Pepper EE | .6 | 0.25% |
| Clary Sage EE | 1.9 | 0.79% |

In certain embodiments of the invention, the topical cream may be incorporated into additional pharmaceutically acceptable therapeutically active agent(s). In one embodiment, the topical cream further comprises an antiseptic compound in a therapeutically effective amount. Another embodiment of the topical cream further comprises an antifungal compound in a therapeutically effective amount. Another embodiment the topical cream further comprises a compound to reduce acne. Another embodiment of the topical cream further comprises a soothing, smoothing, moisturizing or protective agent compound. Additional embodiments of the topical cream further comprise compounds with additional analgesic properties, anti-viral compounds, and UV-ray absorbing compounds. Yet, other embodiments of the invention also comprise compounds or mechanisms to control the timing or release of the therapeutically active components of the topical cream into the body or blood stream. In another embodiment of the invention, the topical cream may not include a base cream, and/or may also comprise a transdermal delivery component (like a transdermal patch) or components sufficient for delivery via a suppository. In yet another embodiment of the present invention, the topical cream may have a veterinary application and may comprises additional components for the treatment of animals. In yet another embodiment of the present invention, the topical cream may have an application as toothpaste or mouthwash and may comprise additional components for treatments in toothpaste-form or mouthwash-form.

In additional embodiments of the present invention, the topical cream utilizes different combinations of terpenes, cannabinoids, and essential oils to treat disease states and conditions, including, but not limited to, spasticity, numbness, radiculopathy, neuropathy, seizures, tremors, lethargy, encephalopathy, focus/attention problems, cognitive issues, anxiety and other mood disorders, insomnia, skin infections, skin lesions, and rashes. In additional embodiments of the present invention, the topical cream may be produced using a different process or method.

The embodiments of the described topical cream and methods are not limited to the embodiments, components, method steps, and materials disclosed herein, as such components, process steps, and materials may vary. Moreover, the terminology employed herein is used to describing exemplary embodiments only and the terminology is not intended to be limiting since the scope of the various embodiments of the present invention will be limited only by the appended claims and equivalents thereof.

Therefore, while embodiments of the invention are described with reference to exemplary embodiments, those skilled in the art will understand that variations and modifications can be affected within the scope of the invention as defined in the appended claims. Accordingly, the scope of the various embodiments of the present invention should not be limited to the above discussed embodiments and should only be defined by the following claims and all equivalents.

1. A topical cream comprising at least one isolated cannabinoid, at least one terpene isolate, and at least one essential oil, wherein the at least one essential oil comprises black pepper essential oil.

2. A topical cream comprising at least one isolated cannabinoid, at least one terpene isolate, at least one essential oil, and a base cream, wherein the at least one essential oil comprises black pepper essential oil.

3. The topical cream of claim 2, wherein the base cream comprises an emulsifier.

4. The topical cream of claim 2, wherein the base cream makes up 65-95% of the topical cream.

5. The topical cream of claim 2, wherein the topical cream also comprises a magnesium chloride lotion.

6. The topical cream of claim 5, wherein the magnesium chloride lotion makes up 10-20% of the topical cream.

7. The topical cream of claim 2, wherein the at least one isolated cannabinoid comprises isolated THC and isolated CBD.

8. The topical cream of claim 7, wherein the concentration of THC compound in the isolated THC is greater than or equal to 5 mg/ml and the concentration of CBD compound in the isolated CBD is greater than or equal to 5 mg/ml.

9. The topical cream of claim 7, wherein the isolated THC makes up 3-7% of the topical cream and the isolated CBD makes up 3-7% of the topical cream.

10. The topical cream of claim 7, wherein the at least one terpene isolate consists of Linalool isolate, Myrcene isolate, and Caryophyllene isolate.

11. The topical cream of claim 10, wherein the Linalool isolate makes up 0.20-2.10% of the topical cream, the Myrcene isolate makes up 0.20-2.10% of the topical cream, and the Caryophyllene isolate makes up 0.20-2.10% of the topical cream.

12. The topical cream of claim 7, wherein the at least one essential oil consists of lavender essential oil, rosemary essential oil, and black pepper essential oil.

13. The topical cream of claim 12, wherein the lavender essential oil makes up 0.20-2.10% of the topical cream, the rosemary essential oil makes up 0.20-2.10% of the topical cream, and the black pepper essential oil makes up 0.20-2.10% of the topical cream.

14. The topical cream of claim 7, wherein the at least one essential oil consists of clary sage essential oil, rosemary essential oil, and black pepper essential oil.

15. The topical cream of claim 14, wherein the clary sage essential oil makes up 0.20-2.10% of the topical cream, the rosemary essential oil makes up 0.20-2.10% of the topical cream, and the black pepper essential oil makes up 0.20-2.10% of the topical cream.

16.-21. (canceled)
